(19) United States
(12) Patent Application Publication
(10) Pub. No.: US 2025/0057526 A1
(43) Pub. Date: Feb. 20, 2025

(54) TISSUE CLOSURE DEVICE

(71) Applicant: Cilag GmbH International, Zug (CH)

(72) Inventors: Jie Zhang, Shanghai (CN); Qinglong Liu, Shanghai (CN)

(21) Appl. No.: 18/719,905

(22) PCT Filed: Dec. 14, 2022
(86) PCT No.: PCT/IB2022/062186

§ 371 Date: Jun. 14, 2024

(30) Foreign Application Priority Data
Dec. 17, 2021 (CN) 202111551922.8

(51) Int. Cl. A61B 17/04

(52) U.S. Cl. CPC A61B 17/0487

(57) ABSTRACT

A tissue closure device is configured to allow a suture to be retained between a proximal end engagement slot and a distal end engagement slot and received in a suture receiving recess prior to entry of the tissue closure device into a tissue, and to cause the suture to be stretched when at least two wings are pivoted from a folded position to an unfolded position, so as to define a suture section that is at least partially parallel to a positioning plane and is configured to be gripped by an instrument. The tissue closure device is configured to carry a suture into a tissue, and such an arrangement omits a needle inserting process for carrying a suture into a tissue.

### TECHNICAL FIELD

The present invention relates to the field of surgical instruments, and in particular to a tissue closure device.

### BACKGROUND OF THE INVENTION

For an existing tissue closure device in surgical instruments, generally, the body of the closure device is penetrated into a human or animal tissue and then a suture is carried by an entry needle into the tissue, after which, the suture is fixed on a distal end structure of the tissue closure device, followed by closure. However, in the process of the entry needle carrying the suture into the tissue, the entry needle is likely to interfere with the body of the closure device or the tissue, which may cause the suture to fall off the entry needle, preventing the suture from being accurately delivered to a predetermined position. And the existing tissue closure device usually cannot accurately place the suture at a predetermined depth.

Therefore, there is a need to provide a tissue closure device that at least partially addresses the aforementioned problems.

### SUMMARY

The present invention provides a tissue closure device, the body of which can be pre-loaded with a suture prior to entering a tissue, so as to carry the suture into the tissue. A depth locating mechanism is unfolded after a distal end of the device enters the tissue, such that the suture is tensioned in a predetermined position to be gripped by an entry needle. Such an arrangement omits a needle inserting process for carrying a suture into a tissue, simplifies the operation process while ensuring that the suture can be accurately arranged in a predetermined position, and ensures the reliability of gripping the suture.

According to an aspect of the present invention, there is provided a tissue closure device comprising:
an elongate body having a longitudinal axis, a sidewall of the elongate body being provided with a suture receiving recess extending from a proximal end to a distal end;
an operating portion disposed at the proximal end of the elongate body, the operating portion being provided with a proximal end engagement slot capable of clamping a suture;
a depth limiting mechanism comprising at least two wings disposed about the longitudinal axis, a proximal end of each wing being pivotally connected to the distal end of the elongate body about a pivot that is perpendicular to the longitudinal axis to allow movement of the wing between a folded position and an unfolded position, wherein the at least two wings together form an extension section of the elongate body when the at least two wings are in the folded position; and the at least two wings extend radially outward relative to the elongate body and together define a positioning plane at top surfaces of the at least two wings when the at least two wings are in the unfolded position, and the depth limiting mechanism is provided with a distal end engagement slot for clamping the suture, the distal end engagement slot being at least partially formed on the wing,
wherein the tissue closure device is configured to allow the suture to be tensioned between the proximal end engagement slot and the distal end engagement slot and received in the suture receiving recess prior to entry of the tissue closure device into a tissue, and to cause the suture to be stretched when the at least two wings are pivoted from the folded position to the unfolded position, so as to define a suture section that is at least partially parallel to the positioning plane and can be gripped by an instrument.

In one embodiment, the distal end engagement slot comprises a first distal end engagement slot and a second distal end engagement slot, the first distal end engagement slot and the second distal end engagement slot being configured to enable the suture to disengage from the first distal end engagement slot but remain engage with the second distal end engagement slot when gripped by the instrument.

In one embodiment, the first distal end engagement slot has a rigid inner slot wall and has an inner dimension greater than the diameter of the suture, and the second distal end engagement slot has an elastic inner slot wall and has an inner dimension less than the diameter of the suture so as to form an interference fit with the suture.

In one embodiment, both the first distal end engagement slot and the second distal end engagement slot are disposed on the wing.

In one embodiment, the first wing engagement slot is a hook-shaped slot comprising an inclined slot extending distally and radially outward simultaneously, the first wing engagement slot having an inner dimension greater than the diameter of the suture, and/or
the wing is provided with a feature made of a flexible material, the second distal end engagement slot being formed on the feature.

In one embodiment, the first distal end engagement slot and the second distal end engagement slot are positioned such that the first distal end engagement slot is radially closer to the central axis than the second distal end engagement slot when the wing is in the unfolded position.

In one embodiment, inner walls of both the first distal end engagement slot and the second distal end engagement slot are rigid, wherein:
an opening direction of the first distal end engagement slot is perpendicular to the longitudinal axis and an included angle between the opening direction and a radial direction with respect to the axis is an acute angle, a proximal end of the first distal end engagement slot being open; and
an opening direction of the second distal end engagement slot is at least partially transverse to the first distal end engagement slot, and the second distal end engagement slot is positioned at a radially outer end of the wing.

In one embodiment, the depth limiting mechanism further comprises a fixing block fixedly connected to the distal end of the elongate body and extending along the longitudinal axis, the fixing block being positioned such that: the at least two wings circumferentially surround the fixing block when the at least two wings are in the folded position, and the fixing block is located distal to a junction of the at least two wings when the at least two wings are in the unfolded position,
wherein the first distal end engagement slot is formed on the fixing block, and the second distal end engagement slot is formed on the at least two wings.

In one embodiment, the fixing block comprises a longitudinal portion extending along the longitudinal axis and a transverse portion perpendicular to the longitudinal axis, the longitudinal portion and the transverse portion together forming a cruciform unit, wherein the first distal end engagement slot is formed at two ends of the transverse portion.

In one embodiment, the second distal end engagement slot is formed at a distal end of each of the wings.

In one embodiment, a distal end of the longitudinal portion of the fixing block is further provided with a third distal end engagement slot, the third distal end engagement slot being configured such that the suture remains engage with the third engagement slot when gripped by the instrument.

In one embodiment, each wing is provided with a window, the window being positioned to allow the suture to extend distally through the window from the suture receiving recess of the elongate body to the first distal end engagement slot.

In one embodiment, the first distal end engagement slot is formed on a distal face at a radially outer end of the transverse portion.

### BRIEF DESCRIPTION OF THE DRAWINGS

To better understand the above and other objectives, features, advantages and functions of the present invention, reference may be made to preferred embodiments shown in the accompanying drawings. The same or similar reference numerals in the drawings refer to the same or similar components. It should be understood by those skilled in the art that the drawings are intended to schematically clarify preferred embodiments of the present invention, without any limitation on the scope of the present invention, and various components in the figures are not necessarily drawn to scale.

FIG. 1 is a schematic perspective view of a tissue closure device according to one preferred embodiment of the present invention;

FIG. 2 is an enlarged schematic perspective view of one wing in FIG. 1;

FIG. 3A is a left view of the wing in FIG. 1;

FIG. 3B is a top view of the wing in FIG. 2;

FIG. 4 is an elevation view of the tissue closure device in FIG. 1 with a suture loaded thereon;

FIG. 5 is a perspective view of a tissue closure device according to another preferred embodiment of the present invention;

FIG. 6 is an enlarged partial view of the portion A in FIG. 5;

FIG. 7 is an enlarged perspective view of one wing in FIG. 6;

FIG. 8 is an elevation view of the tissue closure device in FIG. 5 with a suture loaded thereon;

FIG. 9 is a left view of the tissue closure device in FIG. 5; and

FIG. 10 is an enlarged partial view of the portion B in FIG. 9.

### DETAILED DESCRIPTION

An injector, a body of the injector, and an injection head of the injector according to the present invention will be described in detail below with reference to the drawings. The given below are merely preferred embodiments according to the present invention, and on the basis of the preferred embodiments, those skilled in the art can conceive of other modes capable of implementing the present invention, which also fall within the scope of the present invention.

The present invention provides a tissue closure device for a surgical procedure. First of all, it should be noted that the directional terms and position terms mentioned in the present invention should be understood as relative directions and relative positions. For example, the “axis direction,” “axial direction,” and the like mentioned in the present invention may be understood as the direction of the axis defined by the elongate body; the “radial,” “circumferential,” and the like are radial, circumferential and the like with respect to the axis; “distal end” and “distal” is the direction along the axial direction and away from an operator (e.g., a surgeon), where the direction of distally is shown by D in the drawings; the “proximal end” and “proximal” is the direction along the axial direction and close to the operator (e.g., the surgeon), where the direction of proximally is shown by P in the drawings.

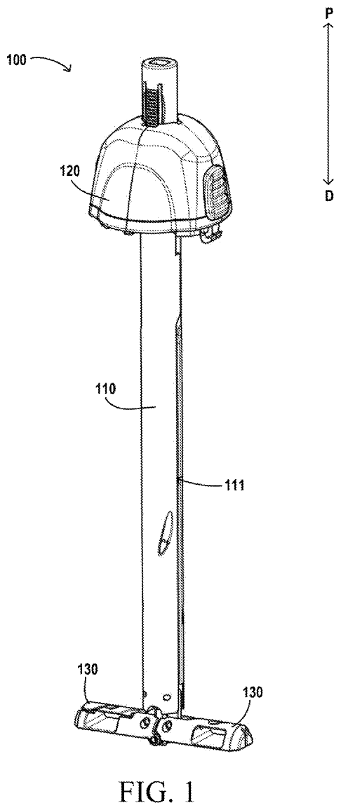

FIGS. 1-4 illustrate one preferred embodiment according to the present invention. Referring first to FIG. 1, a tissue closure device 100 includes an elongate body 110 having a longitudinal axis, an operating portion 120, and a depth limiting mechanism. The sidewall of the elongate body 110 is provided with a suture section receiving recess 111 extending parallel to the longitudinal axis, and the suture section receiving recess may also be disposed to extend from a proximal end to a distal end in a helical-like fashion about the sidewall of the elongate body. The operating portion 120 is disposed at a proximal end of the elongate body 110 and radially protrudes from the elongate body 110, and the operating portion 120 is provided with a proximal end engagement slot capable of clamping a suture 300.

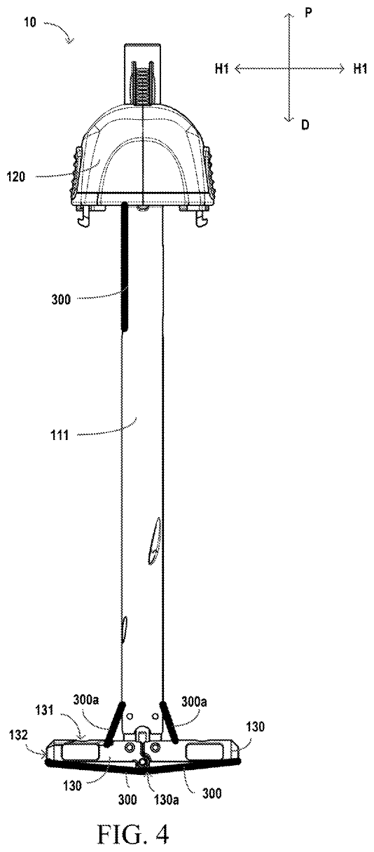

The depth limiting mechanism includes two wings 130 disposed about the longitudinal axis, and a proximal end of each wing 130 is pivotally connected to a distal end of the elongate body 110 about a pivot that is perpendicular to the longitudinal axis to allow movement of the wing 130 between a folded position and an unfolded position. When the two wings 130 are in the folded position, the two wings 130 together form an extension section of the elongate body 110; when the two wings 130 are in the unfolded position (i.e., the state shown in FIGS. 1 and 4), the two wings 130 extend radially outward relative to the elongate body 110 and together define a positioning plane at top surfaces of the two wings 130. The depth limiting mechanism is provided with a distal end engagement slot for clamping the suture 300, and the distal end engagement slot is at least partially formed on the wing 130.

The tissue closure device 100 is configured to allow the suture 300 to be tensioned between the proximal end engagement slot and the distal end engagement slot and received in the suture section receiving recess 111 prior to entry of the tissue closure device 100 into the tissue, and to cause the suture 300 to be stretched when the wing 130 is pivoted from the folded position to the unfolded position, so as to define a suture section 300a that is at least partially parallel to the positioning plane and can be gripped by an instrument (e.g., a needle). FIG. 4 illustrates a schematic view of the tissue closure device 100 with the suture 300 loaded thereon.

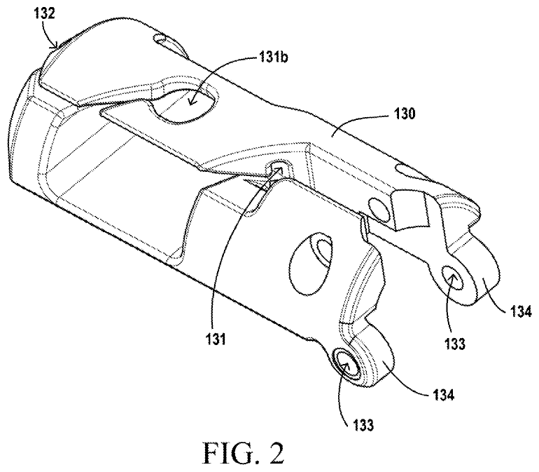

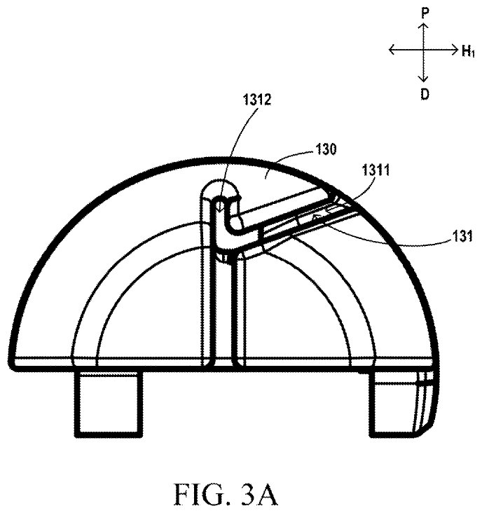

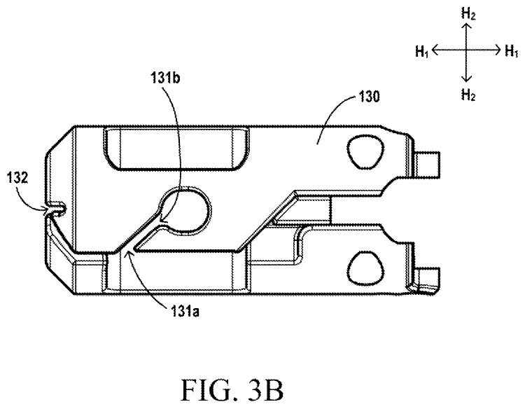

Preferably, with reference to FIGS. 2 and 3, the distal end engagement slot includes a first distal end engagement slot 131 and a second distal end engagement slot 132, and the first distal end engagement slot 131 and the second distal end engagement slot 132 are configured to allow the suture 300 to be gripped by the needle to disengage from the first distal end engagement slot 131 but remain engage with the second distal end engagement slot 132. In this embodiment, the first distal end engagement slot 131 and the second distal end engagement slot 132 are both disposed on the wing 130. And the first distal end engagement slot 131 is radially closer to the central axis than the second distal end engagement slot 132 when the wing 130 is in the unfolded position.

For example, inner walls of the first distal end engagement slot 131 and the second distal end engagement slot 132 may both be rigid. An opening direction of the first distal end engagement slot 131 is perpendicular to the longitudinal axis, and an included angle between the opening direction and a radial direction with respect to the axis is an acute angle, that is, the extension direction of the opening of the first distal end engagement slot 131 has a component extending along a first lateral direction H1 and also a component extending along a second lateral direction H2. The first lateral direction H1 and the second lateral direction H2 are perpendicular to each other and together define a plane perpendicular to the longitudinal axis. The first lateral direction H1 and the second lateral direction H2 may be parallel to two radial directions with respect to the longitudinal axis, respectively. As can be seen from FIGS. 2 and 3, the first distal end engagement slot 131 may be in communication with an open proximal end 131b, and the open proximal end 131b also has an opening 131a for outward withdrawal. The proximal end 131b is formed as an open, approximately circular opening, from where the instrument is allowed to grip the suture section.

With continued reference to FIGS. 2-3B, the second distal end engagement slot 132 may be formed at a radially outer end of the wing 130 and is disposed to open along the first lateral direction H1. The second distal end engagement slot 132 may be supported by silicone or other flexible materials (including elastic materials), such that the second distal end engagement slot 132 can elastically clamp the suture. Alternatively, the inner wall of the second distal end engagement slot 132 may also be rigid. As can also be seen from FIG. 2, the radially innermost side of the wing 130 that is close to the axis is provided with a pivot portion 134, and the pivot portion 134 is formed with a pivot hole 133. The pivot portion 134 protrudes distally relative to the body of the wing 130.

In the end view illustrated in FIG. 3A, it can be seen that the first distal end engagement slot 131 on the wing is formed as a hook-shaped slot, and the hook-shaped slot includes a vertical section 1312 extending parallel to the longitudinal axis and a section 1311 extending along the axial direction and the radial direction simultaneously. Such a unique suture slot design facilitates the insertion of the suture while ensuring that the suture can be released.

Referring to FIG. 4, when the suture 300 is pre-loaded on the tissue closure device 100, a proximal end of the suture 300 will be clamped on the proximal end engagement slot of the operating portion 120, and the suture 300 will extend to the distal end along the suture section receiving recess 111 on the elongate body 110 and be clamped by the first distal end engagement slot 131 and the second distal end engagement slot 132. In particular, the section of the suture 300 that is between the elongate body 110 and the first distal end engagement slot 131 forms a suture section 300a (which can be gripped from the circular opening 131b) which can be gripped by the needle. The most distal end portion of the suture 300 is tensioned between the respective second distal end engagement slots 132 of the two wings 130, and the middle of the portion rests against a distal end 130a of the two wings 130.

In use, the tissue closure device 100 can be pre-loaded with the suture 300 prior to entering a tissue, and the wings 130 are in the folded position prior to entering a tissue. The tissue closure device 100 loaded with the suture 300, with the wings 130 in the folded position, is extended into an incision in the tissue, and when the distal end of the tissue closure device 100 reaches a predetermined depth, the wings 130 can be unfolded by actuation of the operating portion 120. At this time, the suture 300 is also driven by the wings 130 to stretch, and the suture 300 is clamped in the first distal end engagement slot 131 and the second distal end engagement slot 132 respectively. At this time, the suture 300 has a suture section 300a which can be gripped by a needle.

Thereafter, a needle having a hook can be delivered into the tissue, and the hook can catch the suture section 300a of the suture 300 and pull the suture 300 so that the suture 300 is disengaged from the first distal end engagement slot 131. At this time, the suture 300 is still clamped by the second distal end engagement slot 132. Subsequently, the wings 130 can be pivoted back to the folded position by actuation of the operating portion 120 and moved proximally to exit the tissue. In the process, the suture 300 will be disengaged from the second distal end engagement slot 132, such that the distal end of the suture 300 is left within the tissue. After the tissue closure device 100 is removed from the tissue, the suture 300 can be sewn at the surface of the tissue.

In addition to the structures of the first distal end engagement slot and the second distal end engagement slot illustrated in FIGS. 1-4, there are also other possible arrangements for the first distal end engagement slot and the second distal end engagement slot disposed on the wings. In one embodiment, the first distal end engagement slot has a rigid inner slot wall and has an inner dimension greater than the diameter of the suture, and the second distal end engagement slot has an elastic inner slot wall and has an inner dimension less than the diameter of the suture so as to form an interference fit with the suture. For example, the first wing engagement slot is formed as an inclined slot extending distally and radially outward with respect to the central axis simultaneously, the first wing engagement slot having an inner dimension larger than the diameter of the suture, and/or the wing is provided with a silicone block, on which the second distal end engagement slot is formed on.

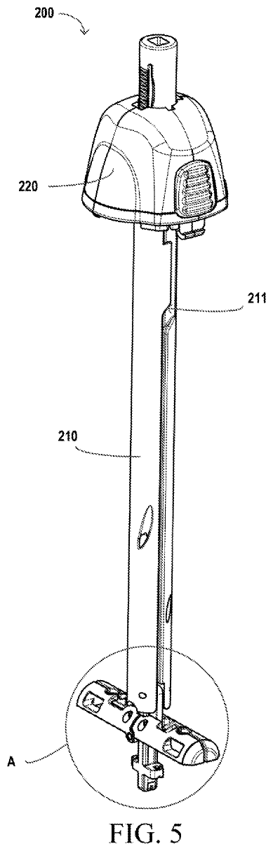

FIGS. 5-10 illustrate another preferred embodiment according to the invention. Referring first to FIG. 5, a tissue closure device 200 includes an elongate body 210 having a longitudinal axis, an operating portion 220, and a depth limiting mechanism. The sidewall of the elongate body 210 is provided with a suture receiving recess 211 extending parallel to the longitudinal axis. The operating portion 220 is disposed at a proximal end of the elongate body 210 and radially protrudes from the elongate body 210, and the operating portion 220 is provided with a proximal end engagement slot capable of clamping a suture 300.

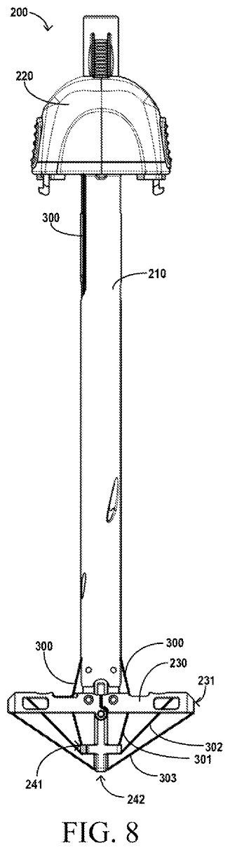

The depth limiting mechanism includes two wings 230 disposed about the longitudinal axis, and a proximal end of each wing 230 is pivotally connected to a distal end of the elongate body 210 about a pivot that is perpendicular to the longitudinal axis to allow movement of the wing 230 between a folded position and an unfolded position. When the two wings 230 are in the folded position, the two wings 230 together form an extension section of the elongate body 210; when the two wings 230 are in the unfolded position (i.e., the state shown in FIGS. 5 and 8), the two wings 230 extend radially outward relative to the elongate body 210 and together define a positioning plane at top surfaces of the two wings 230. The depth limiting mechanism is provided with a distal end engagement slot for clamping the suture 300, and the distal end engagement slot is at least partially formed on the wing 230.

The tissue closure device 200 is configured to allow the suture 300 to be tensioned between the proximal end engagement slot and the distal end engagement slot and received in the suture receiving recess 211 prior to entry of the tissue closure device 200 into the tissue, and to cause the suture 300 to be stretched when the wing 230 is pivoted from the folded position to the unfolded position, so as to define a suture section that is at least partially parallel to the positioning plane and can be gripped by an instrument. FIG. 8 illustrates a schematic view of the tissue closure device 200 with the suture 300 loaded thereon.

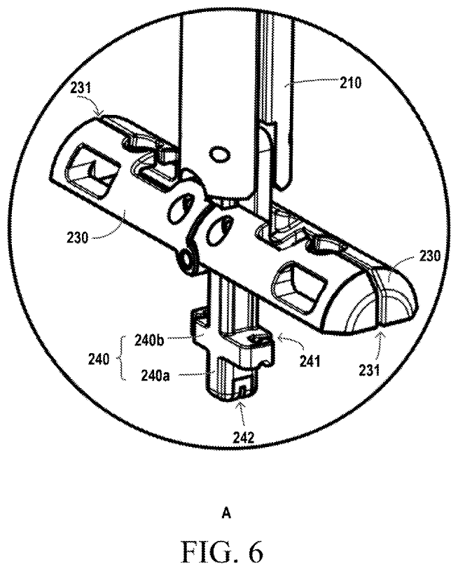

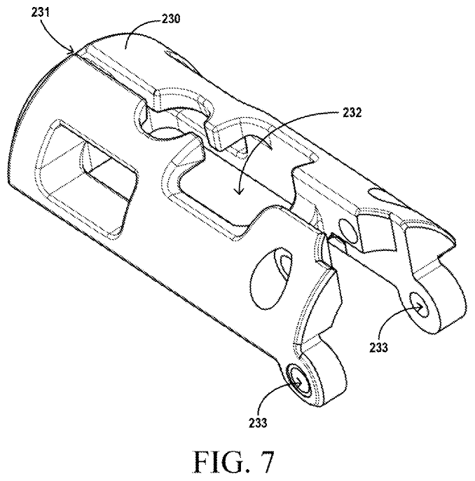

Referring to FIGS. 6 and 7, the depth limiting mechanism further includes a fixing block 240 fixedly connected to the distal end of the elongate body 210 and extending along the longitudinal axis, and the fixing block 240 is positioned such that the two wings 230 circumferentially surround the fixing block 240 when the two wings 230 are in the folded position, and the fixing block 240 is located distal to the two wings 230 when the two wings 230 are in the unfolded position. A first distal end engagement slot 241 is formed on the fixing block 240, and a second distal end engagement slot 231 is formed on the two wings 230. A hole 233 for receiving a pivot is formed on the wing 230.

Preferably, the fixing block 240 includes a longitudinal portion 240a extending along the longitudinal axis and a transverse portion 240b perpendicular to the longitudinal axis, and the longitudinal portion 240a and the transverse portion 240b together form a cruciform unit, where the first distal end engagement slot 241 is formed at two ends of the transverse portion 240b. The second distal end engagement slot 231 is formed at a distal end of each wing 230 (the distal end can be understood as a distal end when the wing 230 is in the folded position).

Further, a distal end of the longitudinal portion 240a of the fixing block 240 is also provided with a third distal end engagement slot 242, and the third distal end engagement slot 242 is configured such that the suture 300 remains engage with the third engagement slot 242 when the suture 300 is gripped by a needle. In this embodiment, the second distal end engagement slot 231 and the third distal end engagement slot 242 are both made of silicone which defines a slot dimension that is less than the width of the suture 300 so that the second distal end engagement slot 231 and the third distal end engagement slot 242 elastically retain the suture 300 in an interference fit manner.

Preferably, referring to FIG. 7, each wing 230 is provided with a window 232, and the window 232 is positioned to allow the suture 300 to extend distally through the window 232 from the suture receiving recess 211 of the elongate body 210 to the first distal end engagement slot 241.

Referring to FIG. 8, a first portion 301 of the suture 300 is tensioned between the elongate body 210 and the first distal end engagement slot 241; a second portion 302 is tensioned between the first distal end engagement slot 241 and the second distal end engagement slot 231; and a third portion 303 is tensioned between the second distal end engagement slot 231 and the third distal end engagement slot 242. In this embodiment, the second portion 302 of the suture 300 is a suture section which can be gripped by the needle.

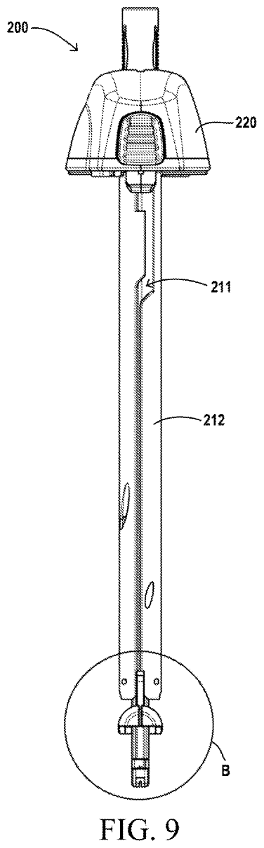

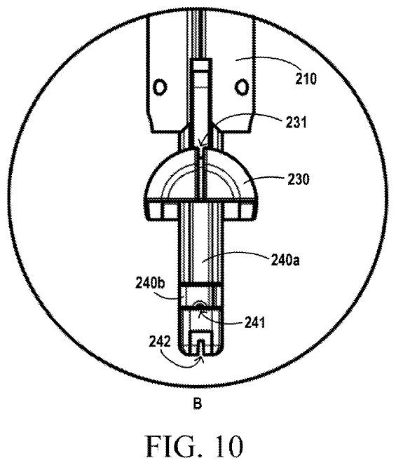

Referring to FIGS. 9 and 10, it can be more clearly seen that the first distal end engagement slot 241 is a shallow slot formed on a distal face at a radially outer end of a transverse portion 242b, the second distal end engagement slot 231 is formed as a slot axially penetrating on a radially outer end face of the wing 230, and the third distal end engagement slot 242 is formed on a distal face of a longitudinal portion 240a and has a greater depth than the first distal end engagement slot 241. Such an arrangement facilitates pre-loading of a suture, and can ensure that the suture is not easy to fall off when the suture is tensioned and released.

In use, the tissue closure device 200 can be pre-loaded with the suture 300 prior to entering a tissue, and the wings 230 are in the folded position prior to entering a tissue. The tissue closure device 200 loaded with the suture 300, with the wings 230 in the folded position, is extended into an incision in the tissue, and when the distal end of the tissue closure device 200 reaches a predetermined depth, the wings 230 can be unfolded by actuation of the operating portion 220. At this time, the suture 300 is also driven by the wings 230 to stretch, and the suture 300 is clamped by the first distal end engagement slot 241, the second distal end engagement slot 231 and the third distal end engagement slot 242 respectively. At this time, the suture 300 has a suture section (the third portion 303) which can be gripped by the needle.

Thereafter, a needle having a hook can be delivered into the tissue, and the hook can catch the suture section of the suture 300 and pull the suture 300 so that the suture 300 is disengaged from the first distal end engagement slot 241. At this time, the suture 300 is still clamped by the second distal end engagement slot 231 and the third distal end engagement slot 242. Subsequently, the wings 230 can be pivoted back to the folded position by actuation of the operating portion 220 and moved proximally to exit the tissue. In the process, the suture 300 will be disengaged from the second distal end engagement slot 231 and the third distal end engagement slot 242, such that the distal end of the suture 300 is left within the tissue. After the tissue closure device 200 is removed from the tissue, the suture 300 can be sewn at the surface of the tissue.

While the number of the wings in the embodiments illustrated in FIGS. 1-8 is only two, in other embodiments not shown, three or more wings may be provided, and the three or more wings may be arranged about the longitudinal axis at equal intervals, that is, any pair of adjacent wings has the same included angle.

The tissue closure device of the present invention can be pre-loaded with a suture prior to entering a tissue, to carry the suture into the tissue. A depth locating mechanism is unfolded after a distal end of the device enters the tissue, such that the suture is tensioned in a predetermined position to be gripped by an entry needle. Such an arrangement omits a needle inserting process for carrying a suture into a tissue, and simplifies the operation process while ensuring that the suture can be accurately arranged in a predetermined position without falling off in an undesired position.

From the above, those skilled in the art will readily recognize that alternative structures of the presently disclosed structures may be used as feasible alternative embodiments, and the presently disclosed embodiments may be combined to create new embodiments, which also fall within the scope of the appended claims.

1. A tissue closure device, the tissue closure device (100, 200) comprising:
an elongate body (110, 210) having a longitudinal axis, a sidewall of the elongate body being provided with a suture receiving recess (111, 211) extending from a proximal end to a distal end;
an operating portion (120, 220) disposed at the proximal end of the elongate body, the operating portion being provided with a proximal end engagement slot configured to clamp a suture;
a depth limiting mechanism comprising at least two wings (130, 230) disposed about the longitudinal axis, a proximal end of each wing being pivotally connected to the distal end of the elongate body about a pivot that is perpendicular to the longitudinal axis to allow movement of the wing between a folded position and an unfolded position, wherein the at least two wings together form an extension section of the elongate body when the at least two wings are in the folded position; and the at least two wings extend radially outward relative to the elongate body and together define a positioning plane at top surfaces of the at least two wings when the at least two wings are in the unfolded position, and the depth limiting mechanism is provided with a distal end engagement slot for clamping the suture, the distal end engagement slot being at least partially formed on the wing,
wherein the tissue closure device is configured to allow the suture to be retained between the proximal end engagement slot and the distal end engagement slot and received in the suture receiving recess prior to entry of the tissue closure device into a tissue, and to cause the suture to be stretched when the at least two wings are pivoted from the folded position to the unfolded position, so as to define a suture section (300a, 302) that is at least partially parallel to the positioning plane and is configured to be gripped by an instrument.

2. The tissue closure device according to claim 1, wherein the distal end engagement slot comprises a first distal end engagement slot (131, 241) and a second distal end engagement slot (132, 231), the first distal end engagement slot and the second distal end engagement slot being configured to enable the suture to disengage from the first distal end engagement slot but remain engaged with the second distal end engagement slot when gripped by the instrument.

3. The tissue closure device according to claim 2, wherein the first distal end engagement slot has a rigid inner slot wall and has an inner dimension greater than the diameter of the suture, and the second distal end engagement slot has an elastic inner slot wall and has an inner dimension less than the diameter of the suture so as to form an interference fit with the suture.

4. The tissue closure device according to claim 2, wherein both the first distal end engagement slot and the second distal end engagement slot are disposed on the wing.

5. The tissue closure device according to claim 4, wherein the first wing engagement slot is formed as a hook-shaped slot comprising an inclined slot extending distally and radially outward simultaneously, the first wing engagement slot having an inner dimension greater than the diameter of the suture.

6. The tissue closure device according to claim 4, wherein the wing is provided with a feature made of a flexible material, the second distal end engagement slot being formed in the feature.

7. The tissue closure device according to claim 4, wherein the first distal end engagement slot and the second distal end engagement slot are positioned such that the first distal end engagement slot is radially closer to the central axis than the second distal end engagement slot when the wing is in the unfolded position.

8. The tissue closure device according to claim 2, wherein inner walls of both the first distal end engagement slot (131) and the second distal end engagement slot (132) are rigid, wherein:
an opening direction of the first distal end engagement slot (131) is perpendicular to the longitudinal axis and an included angle between the opening direction and a radial direction with respect to the axis is an acute angle, a proximal end (131b) of the first distal end engagement slot being open; and
an opening direction of the second distal end engagement slot (132) is at least partially transverse to the first distal end engagement slot, and the second distal end engagement slot is positioned at a radially outer end of the wing.

9. The tissue closure device according to claim 2, wherein the depth limiting mechanism further comprises a fixing block (240) fixedly connected to the distal end of the elongate body and extending along the longitudinal axis, the fixing block being positioned such that: the at least two wings circumferentially surround the fixing block when the at least two wings are in the folded position, and the fixing block is located distal to a junction of the at least two wings when the at least two wings are in the unfolded position,
wherein the first distal end engagement slot (241) is formed on the fixing block, and the second distal end engagement slot (231) is formed on the at least two wings.

10. The tissue closure device according to claim 9, wherein the fixing block comprises a longitudinal portion (240a) extending along the longitudinal axis and a transverse portion (240b) perpendicular to the longitudinal axis, the longitudinal portion and the transverse portion together forming a cruciform unit, wherein the first distal end engagement slot (241) is formed at two ends of the transverse portion (240a).

11. The tissue closure device according to claim 10, wherein the first distal end engagement slot is formed on a distal face at a radially outer end of the transverse portion.

12. The tissue closure device according to claim 9, wherein the second distal end engagement slot (231) is formed at a distal end of each of the wings.

13. The tissue closure device according claim 9, wherein a distal end of the longitudinal portion of the fixing block is further provided with a third distal end engagement slot (242), the third distal end engagement slot being configured such that the suture remains engaged with the third engagement slot when gripped by the instrument.

14. The tissue closure device according to claim 8, wherein each wing is provided with a window (232), the window being positioned to allow the suture to extend distally through the window from the suture receiving recess of the elongate body to the first distal end engagement slot.
